(19) United States
(12) Patent Application Publication
(10) Pub. No.: US 2021/0267226 A1
(43) Pub. Date: Sep. 2, 2021

(54) Method of improving quality of sleep of an organism, a method of improving overall health of an organism, and a method of raising vitality of an organism

(71) Applicant: MEIJI CO., LTD., Tokyo (JP)

(72) Inventors: Seiya MAKINO, Tokyo (JP); Hiroshi KANO, Tokyo (JP)

(73) Assignee: MEIJI CO., LTD., Tokyo (JP)

(21) Appl. No.: 17/186,587

(22) Filed: Feb. 26, 2021

(30) Foreign Application Priority Data
Mar. 2, 2020 (JP) 2020-034639

(51) Int. Cl. A23C 9/123; A23L 29/00; A23L 33/135

(52) U.S. Cl. CPC A23C 9/1234; A23L 33/135; A23L 29/065

(57) ABSTRACT

It is an objective of the present invention to newly create a method of improving quality of sleep and to increase consumer options for such a method. In the method according to the present invention, at least one (i) at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus and (ii) its metabolite or a composition containing the same is ingested by an organism to improve quality of sleep of the organism, to improve overall sense of health of the organism, or to improve vitality of the organism. Here, the lactic acid bacterium of the genus Lactobacillus is preferably Lactobacillus delbrueckii subsp. bulgaricus, and more preferably Lactobacillus delbrueckii subsp. bulgaricus OLL1073R-1(FERM BP-10741).

### TECHNICAL FIELD

A present invention relates to a method of improving quality of sleep of an organism. Additionally, the present invention relates to a method of improving overall health of an organism, and a method of raising vitality of an organism.

### BACKGROUND OF THE INVENTION

There have been proposed “an agent for improving quality of sleep containing bacterium of Lactobacillus casei and/or its treatment as an active ingredient (see, e.g., WO2015/146844)” and “an agent for improving sleep containing lactic acid bacterium containing Lactobacillus helveticus LBH MIKI-020 strains, a fermented product of the lactic acid bacterium, and a cell disrupted product of the lactic acid bacterium (see, e.g., Japanese Published Unexamined Patent Application No. 2016-104802)”.

### PRIOR ART DOCUMENT

### Patent Document

[Patent Document 1] WO2015/146844

[Patent Document 2] Japanese Published Unexamined Patent Application No. 2016-104802

[Patent Document 3] Japanese Translation of PCT International Application No. 2003-517828

### SUMMARY OF THE INVENTION

### Problem to be Solved by the Invention

However, it is difficult to say that there are sufficiently many choices of consumers for the method of using such a sleep quality improving agent. It is an objective of the present invention is to newly create a method of improving quality of sleep of an organism and to increase consumer options for such a method.

### Means for Solving the Problem

In order to solve the above-mentioned problem, as a result of intensive investigation by the present inventors, the present invention has been completed by finding that quality of sleep of an organism is significantly improved when at least one (i) at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus (hereinafter, sometimes referred to as the “specified Lactobacillus group”) and (ii) its metabolite or a composition containing the same is ingested into the organism.

In other words, the present invention is as follows.
(1) A method for improving quality of sleep of an organism by ingesting to the organism at least one (i) at least one lactic acid bacterium of genus Lactobacillus selected from the specified Lactobacillus group and (ii) its metabolite or a composition containing the same. Here, the term “metabolite” as used herein means a substance or the like produced as a result of fermentation of milk by lactic acid bacterium. Also, the term “organism” as used herein is, for example, an animal (including a human) such as a mammal. The term “composition” as used herein includes a product that can be ingested by animals (including humans), such as formulation e.g. liquid food, supplement, food additive and the like, food and beverage (excluding animal and plant themselves), and food and beverage composition (including processed food and beverage). Furthermore, the term “food and drink” as used herein includes fermented milk. Also, the term “improved sleep quality” as used herein means a decrease in the total score on the Pittsburgh Sleep Questionnaire. The higher the total score on the Pittsburgh Sleep Questionnaire, the more likely the sleep disturbance is to occur. That is, the “method for improving quality of sleep” can be referred to as “a method for lowering the total score of the Pittsburgh Sleep Questionnaire”, “a method for preventing sleep disturbance”, or “a method for resolving sleep disturbance”. Furthermore, the results of the inventors' study indicate that this method of improving quality of sleep further improves quality of sleep of particularly healthy person, i.e., a person not suffering from sleep disturbance, or suppresses deterioration of quality of sleep of a healthy person, i.e., a person not suffering from sleep disturbance.
(2) The method according to (1), wherein the metabolite is an extracellular polysaccharide of the lactic acid bacterium.
(3) The method according to (1) or (2), wherein the lactic acid bacterium of the genus Lactobacillus is Lactobacillus delbrueckii subsp. bulgaricus OLL1073R-1(FERM BP-10741).

Furthermore, the present inventors have completed another invention by finding that (i) overall health of an organism is improved, (ii) vitality of the organism is raised when at least one (i) at least one lactic acid bacterium of genus Lactobacillus selected from the specified Lactobacillus group and (ii) its metabolite or a composition containing the same is ingested into the organism.

That is, the other invention is as follows.
(4) A method for improving overall health of an organism or for raising vitality of an organism by ingesting to the organism at least one (i) at least one lactic acid bacterium of genus Lactobacillus selected from the specified Lactobacillus group and (ii) its metabolite or a composition containing the same. Here, the term “metabolite” as used herein means a substance or the like produced as a result of fermentation of milk by lactic acid bacterium. Also, the term “organism” as used herein is, for example, an animal (including a human) such as a mammal. The term “composition” as used herein includes a product that can be ingested by animals (including humans), such as formulation e.g. liquid food, supplement, food additive and the like, food and beverage (excluding animal and plant themselves), and food and beverage composition (including processed food and beverage). Furthermore, the term “food and drink” as used herein includes fermented milk.

Incidentally, the improvement of the overall health and the raise of the vitality can be evaluated by performing MOS Short-Formed 8-item Health Survey (SF-8) on the subject. MOS Short-Formed 8-item Health Survey (SF-8) uses a SF-8 questionnaire, which is copyrighted by iHOPE International Corporation. Under such circumstances, this questionnaire can be obtained by the user registering with iHOPE International Corporation.
(5) The method according to (4), wherein the metabolite is an extracellular polysaccharide of the lactic acid bacterium.
(6) The method according to (4) or (5), wherein the lactic acid bacterium of the genus Lactobacillus is Lactobacillus delbrueckii subsp. bulgaricus OLL1073R-1(FERM BP-10741).

Here, at least one (i) at least one lactic acid bacterium of genus Lactobacillus selected from the specified Lactobacillus group and (ii) its metabolite or a composition containing the same can exhibit all functions of improving quality of sleep, improving overall health and raising vitality at a time.

### Effect of the Invention

According to the present invention, quality of sleep and overall health of an organism can be remarkably improved and vitality can be raised by ingesting to the organism at least one (i) at least one lactic acid bacterium of genus Lactobacillus selected from the specified Lactobacillus group and (ii) its metabolite or a composition containing the same. In particular, when the composition is fermented milk, it can greatly contribute to improvement of quality of sleep, improvement of overall health and raise of vitality since the composition can be easily ingested by familiarity.

### Mode for Carrying Out the Invention

The present invention employs at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus (hereinafter, sometimes referred to as the “specified Lactobacillus group”). As a result of extensive studies by the present inventors, it has been clarified that at least one of this lactic acid bacterium and its metabolite, or a composition containing the same, can improve quality of sleep, overall health, or can raise the vitality. The term “composition” as used herein includes a product that can be ingested by animals (including humans), such as formulation e.g. liquid food, supplement, food additive and the like, food and beverage (excluding animal and plant themselves), and food and beverage composition (including processed food and beverage) and it is particularly preferable that the composition is fermented milk. Examples of a method for preparing fermented milk include “a method in which a lactic acid bacterial starter containing a lactic acid bacterium described above is added to a raw milk of a raw material milk after sterilizing and cooling the raw milk, and the raw milk containing the lactic acid bacterium starter is fermented at a fermentation temperature and a fermentation time so as to have a predetermined lactic acidity” As the lactic acidity, for example, 0.6 to 1.2% by mass is exemplified. As the fermentation temperature, for example, 40 to 45° C. is exemplified. As the fermentation time, for example, 2 to 12 hours are exemplified.

The term “lactic acid bacterium” in the present invention is a generic term for all bacteria classified as lactic acid bacterium, and is not limited by bacterial species, strains and the like. It should be noted that the lactic acid bacterium may be classified into either plant origin or animal origin depending on their origin, but in the present invention, any of the lactic acid bacteria derived from plants and the lactic acid bacterium derived from animals can be used. As a strain of the lactic acid bacterium of the present invention, 1 or 2 or more strains selected from lactic acid bacterium of the genus Lactobacillus are preferred because they have sufficient dietary experience in fermented milk such as yogurt. The lactic acid bacterium of the genus Lactobacillus are preferably at least one selected from the specified Lactobacillus group, more preferably Lactobacillus delbrueckii subsp. bulgaricus, and among these, “ Lactobacillus delbrueckii subsp. bulgaricus OLL1073R-1 (Deposit No.: FERM BP-10741)”.

In addition, in such a case, it is preferable to use a thermophilus bacterium or a Streptococcus thermophilus in combination with a lactic acid bacterium of the genus Lactobacillus as a lactic acid bacterium starter from the viewpoints of productivity and palatability. Further, when the lactic acid bacterium of the genus Lactobacillus is Lactobacillus delbrueckii subsp. bulgaricus OLL1073R-1 (Deposit No.: FERM BP-10741), it is more preferable that the thermophilus bacterium is Streptococcus thermophilus 1131 or Streptococcus thermophilus OLS3059 (Deposit No.: FERM BP-10740). Furthermore, lactic acid bacteria other than lactic acid bacterium of the genus Lactobacillus and thermophilus bacterium and fermenting microorganisms such as Bifidobacterium and yeast may be added as a lactic acid bacterial starter or for the purpose of imparting functionality.

Here, “Lactobacillus delbrueckii subsp. bulgaricus OLL1073R-1” has been deposited with the International Patent Organism Depositary of the National Institute of Advanced Industrial Science and Technology at the deposit number FERM BP-10741 (deposit date: Nov. 29, 2006). “Streptococcus thermophilus OLS3059” has been deposited with the International Patent Organism Depositary of the National Institute of Advanced Industrial Science and Technology at the Deposit No. FERM BP-10740 (deposit date: Nov. 29, 2006). This “Lactobacillus delbrueckii subsp. bulgaricus OLL1073R-1” can also be obtained from Meiji Probio Yogurt R-1 manufactured by Meiji Co., Ltd. The Streptococcus thermophilus 1131 is available from Meiji Bulgaria Yogurt LB81 manufactured by Meiji Co., Ltd.

Incidentally, the term “fermented milk” as described above refers to a product obtained by fermenting milk, and includes, for example, but is not limited to, “fermented milk”, “lactic acid bacterial beverage”, “milk beverage”, “Natural Cheese”, and the like, which are defined by the Ministerial Ordinance on Milk and Milk products Concerning Compositional Standards , etc. (Ordinance of the Ministry of Milk, etc.). For example, fermented milk refers to “fermented milk” defined by the Ordinance of the Ministry of Milk, etc.; i.e., a solid (hard type), gelatinous (soft type) or liquid (drink type) obtained by fermenting milk such as raw milk, cow milk, special milk, raw goat milk, sterilized goat milk, raw sheep milk, ingredient-adjusted milk, low-fat milk, fat-free milk and processed milk or milk containing an equivalent or higher non-fat milk solids by lactic acid bacterium or yeast, or frozen of these. Note that the fermented milk is not limited to the above-mentioned. In the fermented milk of the present invention, the range of the concentration of the non-fat milk solids is preferably, for example, 4.0% to 20.0%, more preferably 6.0% to 15.0%, and still more preferably 8.0% to 11.0% Furthermore, the concentration of the milk fat is preferably, for example, 0.2% to 4.0%, more preferably 0.3% to 3.0%, and still more preferably 0.4% to 2.0%.

Typical examples of fermented milk include yogurt. The International Standard defined by the Food and Agriculture Organization of the United Nations (FAO)/World Health Organization (WHO) also stipulates that “products called yogurts are produced from dairy products such as milk and skimmed milk by the lactic acid fermentative action of both Streptococcus thermophilus and Lactobacillus delbrueckii ssp. bulgaricus, and that the two aforementioned bacteria are abundantly viable in the final products.” In the present invention, “yogurt” includes yogurt as defined by the above FAO/WHO. The yogurt includes, for example, plain yogurt, hard yogurt (set-type yogurt), soft yogurt, drink yogurt, and the like. In the present invention, drink yogurt is particularly preferred from the viewpoint of ease of drinking.

By setting at least one of lactic acid bacterium and metabolite thereof according to the present invention or a composition containing the same in a form of a 1 package in an appropriate amount for 1 ingestion, at least one of lactic acid bacterium and metabolite thereof according to the present invention or a composition containing the same can be taken appropriately and easily, which is preferable from the viewpoint of usability. Although the amount appropriate for one intake is dependent on individual differences or variations, quality of sleep to be improved, degree of overall health, degree of vitality, quantity of meal and physical condition and the like, the amount appropriate for one intake is preferably in the range of 50 mL to 200 mL per one time, and more preferably in the range of 80 mL to 150 mL, and more preferably in the range of 100 mL to 120 mL, for example, when the composition is fermented milk containing 8.0% non-fat milk solids. Alternatively, it is preferably within a range of 50 g to 200 g per 1 times, more preferably within a range of 80 g to 150 g, and still more preferably within a range of 100 g to 120 g.

In the present invention, “the form of one package” includes all forms, for example, a general packaging form such as a container with a lid, a bottle with a cap, a single bag, a pouch, a tube and the like. In the present invention, it is possible to clarify the use of at least one of the lactic acid bacterium and the metabolite thereof according to the present invention, or the composition containing the same, by describing the use, the efficacy, the method of ingestion and the like on package containing each individual package or a plurality of individual packages, and/or by enclosing a description or the like into the package, and/or by posted the description or the like, such as a brochure or the like.

At least one of the lactic acid bacterium and their metabolite of the present invention, or a composition containing the same, is preferably ingested continuously for 2 weeks or longer, more preferably for 4 weeks or longer, still more preferably for 6 weeks or longer, still more preferably for 8 weeks or longer, still more preferably for 10 weeks or longer, still more preferably for 12 weeks or longer, still more preferably for 16 weeks or longer, still more preferably for 24 weeks or longer, and particularly preferably for 36 weeks or longer from the viewpoint of improvement of quality of sleep and overall health, and raise of vitality. When the composition according to the present invention is fermented milk, the upper limit of the intake period is not particularly limited and can be continued permanently since the composition has sufficient dietary experience and can be safely ingested, but is, for example, 60 weeks or less if the upper limit is forcibly provided. The upper limit may be, for example, 120 weeks or less, 100 weeks or less, or 80 weeks or less depending on individual differences, such as quality of sleep to be improved, degree of overall health, degree of vitality, and quantity of meal and physical condition.

At least one of the lactic acid bacterium and the metabolite thereof according to the present invention, or a composition containing the same, can improve quality of sleep and overall health, and can raise vitality by being ingested. Here, even if the lactic acid bacterium in the composition is a live bacterium or a dead bacterium, the composition can exhibit the same improving effect.

Incidentally, it is preferable that at least one of the lactic acid bacterium and the metabolite thereof according to the present invention, or a composition containing the same, be labeled with a description such as its use, efficacy, function, type of active ingredient, and method of use. “Labeling” as used herein includes all labeling to inform consumers of the description of the above effects. This labeling may be any labeling capable of recalling or analogizing the labeling content described above, and may include any labeling regardless of the purpose of the labeling, the content of the labeling, the object, the medium to be labeled, or the like. For example, the description may be labeled on a package or container of a product, the description may be displayed or distributed on an advertisement or price list or transaction document related to the product, or the information containing the description may be provided by an electromagnetic (Internet or the like) method.

When at least one of the lactic acid bacterium and the metabolite thereof according to the present invention or a composition containing the same is a product for improving quality of sleep, it is preferable that a product obtained by packaging a product for improving quality of sleep is labeled, for example, “improvement in quality of sleep” or the like. In addition, when at least one of the lactic acid bacterium and the metabolite thereof according to the present invention or a composition containing the same is a product for improving overall health, it is preferable that a product obtained by packaging a product for improving overall health is labeled, for example, “improvement in overall health” or the like. In addition, when at least one of the lactic acid bacterium and the metabolite thereof according to the present invention or a composition containing the same is a product for raising vitality, it is preferable that a product obtained by packaging a product for raising vitality is labeled, for example, “raise in vitality” or the like.

Note that the words used for performing the above-described labeling are not limited to the above-described example, and may be words having the same meaning as the above-described example. Such words may include, for example, the following words for consumers: “It improves quality of sleep”, “It raises quality of sleep”, “It prevents decrease quality of sleep”, “It reduces overall score on the Pittsburgh Sleep Questionnaire”, “It changes overall score on the Pittsburgh Sleep Questionnaire into score meaning improvement of quality of sleep”, “It promotes sleep”, “It prevents sleep disturbance”, “It helps in dissolving sleep disturbance”, “It makes it easier to fall asleep”, “It falls into a profound sleep”, “It improves overall health”, “It maintains overall health,” “It reduces or prevents decreased overall health”, “It raises vitality”, “It reduces or prevents decreased vitality,” “It maintains or improves vitality” and “It allows vitality to be improved”.

Note that the fermented milk described above can also be made into a form such as a special use food, an integrated nutritional food, a dietary supplement, a specific health food, a functional labeled food, a processed food, or the like. Further, the fermented milk may be blended into a composition such as a beverage other than drink yogurt or a liquid food.

The present invention has been found, according to the examples described later, by the discovery that a composition containing lactic acid bacterium of the specific genus Lactobacillus and its metabolite has an effect of improving quality of sleep and overall health and an effect of raising vitality by comparing a subject who ingests a beverage containing lactic acid bacterium of the specific genus Lactobacillus and a subject who does not ingest the same beverage. Accordingly, the present invention also provides lactic acid bacterium of the genus Lactobacillus and metabolite thereof having these effects. At this time, the intake of lactic acid bacterium of the genus Lactobacillus per day for the purpose of obtaining these effects varies depending on the age, sex and the like, but can be appropriately determined by a person skilled in the art. The daily intake is preferably 10⁶ cfu or more, more preferably 10⁷ cfu or more, and more preferably 10⁸ cfu or more as lactic acid bacterium of the genus Lactobacillus. The upper limit of the intake is not particularly limited, and is, for example, 10¹² cfu.

The composition according to the present invention includes not only a bacterial cell itself but also a component derived from a bacterial cell such as a fungus body milled product. The metabolite of lactic acid bacterium according to the present invention includes metabolite by culturing or fermenting lactic acid bacterium. For example, the lactic acid bacterium of the genus Lactobacillus, Lactobacillus delbrueckii subsp. bulgaricus OLL1073R-1 (Deposit No.: FERM BP-10741), is known to produce extracellular polysaccharides (EPSs) during its culture, and it is also included in the present invention to use this extracellular polysaccharide. In the present invention, the lower limit of the intake per 1 day of these extracellular polysaccharides is 100 μg, preferably 500 μg, more preferably 1.0 mg, and still more preferably 2.0 mg. The upper limit is not particularly limited, but is, for example, 200 mg, preferably 100 mg, more preferably 70 mg, still more preferably 30 mg, and still more preferably 8.0 mg.

### EXAMPLE

Hereinafter, the present invention will be described more specifically on the basis of example. Note that this example is not intended to limit the present invention.

The following test was carried out using the Meiji Probio Yogurt R-1 drink type (the drink yogurt obtained by fermenting milk with Lactobacillus delbrueckii subsp. bulgaricus OLL1073R-1(FERM BP-10741)) manufactured by Meiji Co., Ltd. Since the launch in 2009, this yogurt has been sold sufficiently, and no serious adverse reactions considered to be caused by this product have been reported in the past.

### Test

A parallel group comparison study was conducted between the drink yogurt ingesting group and the non- ingesting group. Hereinafter, the test conditions will be described in detail.

### (1) Subjects

Subjects in this study were those who met the following conditions.
A) Women aged 20 or older with medical and welfare-related qualifications working at medical institutions in Ehime Prefecture (excluding physicians and dentists)
B) Persons who can voluntarily agree to participate in the study after thoroughly understanding the purpose and content of the study.

Those who met the above conditions A) and B) and met any of the following conditions were excluded from the study subjects.
a) People who are pregnant or who are scheduled to become pregnant during the planned study period
b) People with milk allergy
c) People with lactose intolerance
d) People who has received guidance on calorie restriction from a physician.
e) Participants in other clinical trials within the past 3 months
f) Other people deemed ineligible by the responsible physician for this study

This study was conducted with the approval of the Clinical Research Ethics Committee of Ehime University Hospital (Approval No.: Ehime University Medical School Hospital 167015) and the Clinical Trials Review Board of Meiji Co., Ltd. (Review No.: 89). Subjects were recruited at 23 medical institutions in Ehime Prefecture. As a result, 936 people (aged 20-71 years) were selected as subjects. The subjects were then randomly assigned to 473 people (39.3±11.44 years old) in the yogurt ingesting group and 463 people (39.4±11.4 years old) in the non-ingesting group. In this assignment, the number and average age of both groups in each facility were adjusted to be equal.

### (2) Preliminary Preparation

Subjects were instructed not to take yogurt, lactic acid bacterial beverage and supplement containing lactic acid bacterium and the like until random assignment results were obtained after consenting to participate in the study. After random assignment into both groups, the yogurt ingesting group was instructed to consume the above-mentioned drink yogurt once a day for 16 weeks and to refrain from consuming other yogurt, lactic acid bacterial beverage and the like. On the other hand, the non-ingesting group was instructed to refrain from ingesting all yogurts, lactic acid bacterial beverage and the like for 16 weeks.

### (3) Test Details and Results

(3-1) Pittsburgh Sleep Questionnaire

The Pittsburgh Sleep Questionnaire (self-administered questionnaire) was distributed to subjects before the start of the study and after the end of the study (16 weeks after the start of the study). The Pittsburgh Sleep Questionnaire asked subjects' subjective measures for seven items: “quality of sleep”, “start time of sleep”, “sleep duration”, “sleep efficiency”, “difficulty sleeping”, “use of sleeping agents” and “difficulty waking during daytime”. The seven items described above were then scored to calculate the total score for each subject. The higher the total score, the more the sleep disturbance is determined to have occurred. The Pittsburgh Sleep Questionnaire is explained in detail in 1) Tyler R S, Baker L J. Difficulties experienced by tinnitus sufferers. J Speech Hear Disor1983; 48: 150-154, 2) Buysse D J, Reynold C F 3rd, et al. The Pittsburgh Sleep Quality Index: anew instrument for psychiatric practice and research. Psychiatry Res. 1989; 28: 193-213; 3) Doi Yuki, Preparation of Japanese Version of the Pittsburgh Sleep Questionnaire: Psychiatric Therapy 1998: 3: 755-763, 4) ZungW. W. K., Kazuhiko Fukuda, et al. SDS User's Guide 1983 Kyoto: Sankyo-Bo; 5) Spielberger C. D., Kiminobu Mizuguchi, et al. STAI User's Guide 1991 Kyoto: Sankyo-Bo, et al.

Statistical analysis of the subject's responses to the Pittsburgh Sleep Questionnaire showed significant improvements in quality of sleep in the yogurt ingesting group (P=0.007, mean change in the yogurt ingesting group: 5.50→5.03, mean change in the non-ingesting group: 5.33→5.22).
(3-2) MOS Short-Formed 8-item Health Survey (SF-8)

SF-8 questionnaires (self-administered questionnaires consisting of all eight questions) were distributed to subjects before the start of the study and after the end of the study (16 weeks after the start of the study) to have them make response to the questionnaires. SF-8 questionnaire asked a measure of subjective physical and mental well-being. SF-8 scales and evaluations are as follows. In each scale, 50 points are regarded as national standard points, and the higher the score, the better the quality of life. Statistical analysis of the results of the above questionnaires revealed a significant improvement in overall health (P=0.02) and vitality (P=0.01) in the yogurt ingesting group compared with the non-ingesting group.
a) Physical function: How much physical activity and exercise can people do?
b) Daily role function (physical): Is there a physical problem in daily life?
c) Pain in the body: Is there any problem that the body was too painful to work?
d) Overall health: How do people subjectively feel own overall health status?
e) Vitality: How well or tired?
f) Social Life Function: Is there any problem of communication with the surroundings for physical and psychological reasons?
g) Daily role function (mental): Is there a mental problem in daily life?
h) Mental health: how much do people have ups and downs in their mind?

### INDUSTRIAL APPLICABILITY

The method according to the invention makes it possible to improve quality of sleep of an organism, to improve overall health of an organism and to raise vitality of an organism. Fermented milk, in particular, is a food with extensive dietary experience and can be easily ingested at home, thus providing an effective measure for improving quality of sleep, improving overall health, and maintaining vitality, and greatly contributing to improving quality of sleep, overall health and to raising vitality, and is very useful.

### ACCESSION NO.

FERM BP-10741

FERM BP-10740

1. A method of improving quality of sleep of an organism by ingesting to the organism at least one (i) at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus and (ii) its metabolite or a composition containing the same.

2. The method according to claim 1, wherein the metabolite is an extracellular polysaccharide of the lactic acid bacterium.

3. The method according to claim 1, wherein the composition is fermented milk.

4. The method according to claim 1, wherein the lactic acid bacterium of the genus Lactobacillus is Lactobacillus delbrueckii subsp. bulgaricus OLL1073R-1(FERM BP-10741).

5. A method of improving overall health of an organism by ingesting to the organism at least one (i) at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus and (ii) its metabolite or a composition containing the same.

6. A method of improving vitality of an organism by ingesting to the organism at least one (i) at least one lactic acid bacterium of genus Lactobacillus selected from the group consisting of Lactobacillus delbrueckii, Lactobacillus delbrueckii subsp. bulgaricus, Lactobacillus gasseri, Lactobacillus paragasseri, Lactobacillus rhamnosus and Lactobacillus acidophilus and (ii) its metabolite or a composition containing the same.

7. The method according to claim 2, wherein the composition is fermented milk.

8. The method according to claim 2, wherein the lactic acid bacterium of the genus Lactobacillus is Lactobacillus delbrueckii subsp. bulgaricus OLL1073R-1(FERM BP-10741).

9. The method according to claim 3 wherein the lactic acid bacterium of the genus Lactobacillus is Lactobacillus delbrueckii subsp. bulgaricus OLL1073R-1(FERM BP-10741).

10. The method according to claim 7, wherein the lactic acid bacterium of the genus Lactobacillus is Lactobacillus delbrueckii subsp. bulgaricus OLL1073R-1(FERM BP-10741).
